SEQUENCE LISTING (12 sequences)

SEQ ID NO: 1 - DNA, 58 bp, Artificial
  Synthetic DNA
tttggtaccatggatgacgactcctacgtgcccgggtttgaggactcggaggcgggtt

SEQ ID NO: 2 - DNA, 48 bp, Artificial
  Synthetic DNA
gcggaattcgtagctgtccttaggccaaggccggttacgctgccagtt

SEQ ID NO: 3 - DNA, 36 bp, Artificial
  Synthetic DNA
ggggaattcgccaaagaaagggagcgggtggagaac

SEQ ID NO: 4 - DNA, 51 bp, Artificial
  Synthetic DNA
tttgaattcttactgagttttgaccatgcgacggatgtagaaacgcattct

SEQ ID NO: 5 - DNA, 7242 bp, Artificial
  Synthetic DNA
atggatgacgactcctacgtgcccgggtttgaggactcggaggcgggttcagccgactcctacaccagccgcccctctctggactcagacgtttccctggaggaggaccgggagagtgcccggcgagaagtggagagtcaggctcagcagcagctggaaagagccaagcacaaacctgtggcatttgctgtgaggaccaatgtcagctactgtggagttctggatgaggaatgcccagtccagggctctggagtcaacttcgaggccaaagattttctgcacattaaagagaagtacagcaatgactggtggatcgggaggctagtgaaagaaggtggcgatattgccttcatccccagcccccaacgcctggagagcatccggctcaaacaggaacagaaggccaggagatccgggaacccttccagcctgagtgacattggcaaccgacgttcccctcctccatctctagccaagcagaagcaaaagcaggcggaacatgtccccccgtatgatgtggtgccctccatgcggcctgtggtgctggtgggaccctctctgaaaggttatgaggtcacagacatgatgcagaaggctctcttcgacttccttaaacacaggtttgatggcaggatctccatcacccgcgtcacggctgacctctcactggccaagcgctctgtgctcaacaatcctggcaagaggaccatcatcgagcgctcttctgcccgctccagcattgctgaggtgcagagtgagattgagcgcatattcgagctggccaaatccctgcagctagtggtgttggatgctgacaccatcaaccacccagcacagctagccaagacctcactggcccccatcatcgtcttcgtcaaagtgtcctcgccaaaggtactgcagcgactgatccgctccagggggaagtcccagatgaagcacctcactgtacagatgatggcatatgataagctggttcagtgcccacctgagtcatttgatgtgattctggatgagaaccagctggacgacgcctgtgagcacctagctgaatacctagaggtttactggcgcgctacccaccacccagcaccgggccccgggatgctgggtccgcccagtgccatccctggacttcagaaccagcagctgctgggggagcgaggtgaggagcattcacccctggagcgggacagtttgatgccctcggatgaggccagtgagagctcccgccaggcctggaccggatcttcacagcgcagctcccgccatctggaggaggactatgcagatgcctaccaggacctgtaccagcctcaccgtcaacacacctcggggctacccagtgctaacgggcatgacccccaagaccggctcctagcccaggactcggagcatgaccacaatgaccggaactggcagcgtaaccggccttggcctaaggacagctacgaattcgccaaagaaagggagcgggtggagaaccggcgcgcattcctgaagctgcggcggcagcagcagattgaacgcgagctcaacgggtacatggagtggatctcaaaagcagaagaggtgatcctcgcagaggacgagaccgacgtggagcagagacatccctttgatggagctctgcggagagccactatcaagaagagcaagacggacctgctccacccagaggaggcggaggatcagctggccgacatcgcctccgtggggtctccctttgcccgagccagcattaaaagtgccaagctggagaactcgagttttttccacaaaaaagagaggagaatgcgtttctacatccgtcgcatggtcaaaactcagtaagaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtacc

SEQ ID NO: 6 - DNA, 39 bp, Artificial
  Synthetic DNA
agccgtacgcgatcgcatctctagccaagcagaagcaaa

SEQ ID NO: 7 - DNA, 42 bp, Artificial
  Synthetic DNA
cccgttaaccccactagtctgagttttgaccatgcgacggat

SEQ ID NO: 8 - DNA, 33 bp, Artificial
  Synthetic DNA
gggactagtatggtgagcaagggcgaggagctg

SEQ ID NO: 9 - DNA, 34 bp, Artificial
  Synthetic DNA
cccgttaactgccgagagtgatcccggcggcggt

SEQ ID NO: 10 - DNA, 7242 bp, Artificial; identical to SEQ ID NO: 5
  Synthetic DNA

SEQ ID NO: 11 - protein, 10 aa, Artificial
  Synthetic Peptide
  MISC_FEATURE (3)..(3): Xaa = any amino acid
  MISC_FEATURE (4)..(4): Xaa = any amino acid
  MISC_FEATURE (6)..(6): Xaa = any amino acid
  MISC_FEATURE (8)..(8): Xaa = any amino acid
QQXXRXLXGY

SEQ ID NO: 12 - protein, 18 aa, Artificial
  Synthetic Peptide
  MISC_FEATURE (3)..(3): Xaa = any amino acid
  MISC_FEATURE (5)..(5): Xaa = any amino acid
  MISC_FEATURE (6)..(6): Xaa = any amino acid
  MISC_FEATURE (8)..(8): Xaa = any amino acid
  MISC_FEATURE (11)..(11): Xaa = any amino acid
  MISC_FEATURE (12)..(12): Xaa = any amino acid
  MISC_FEATURE (15)..(15): Xaa = any amino acid
  MISC_FEATURE (16)..(16): Xaa = any amino acid
  MISC_FEATURE (17)..(17): Xaa = any amino acid
QQXEXXLXGYXXWIXXXE

(19) United States
(12) Patent Application Publication
(10) Pub. No.: US 2007/0141665 A1
(43) Pub. Date: Jun. 21, 2007

(54) Chimeric protein for the screening of agonists and antagonists of cell signalling pathways that are dependent on g-protein-coupled receptors

(75) Inventors: Michel De Waard, Saint-Christophe/Guiers (FR); Alain Dupuis, Grenoble (FR); Didier Grunwald, Saint Egreve (FR); Guillaume Sandoz, Grenoble (DE)

Correspondence Address: OBLON, SPIVAK, MCCLELLAND, MAIER & NEUSTADT, P.C., 1940 DUKE STREET, ALEXANDRIA, VA, 22314, US

(73) Assignees: COMMISSARIAT A L'ENERGIE ATOMIQUE, Paris (FR); INSTITUT NATIONAL DE LA SANTE ET DE LA RECHER. MED, Paris (FR)

(21) Appl. No.: 10/540,247

(22) PCT Filed: Dec. 22, 2003
(86) PCT No.: PCT/FR03/03860

§ 371 Date: Jun. 5, 2006

(30) Foreign Application Priority Data
Dec. 23, 2002 (FR) 02/16576

(51) Int. Cl. C07K 14/705; C07H 21/04; C12P 21/06

(52) U.S. Cl. 435/69.1; 435/320.1; 435/325; 530/350; 536/23.5

(57) ABSTRACT

The invention relates to a chimeric protein which is derived from a high-threshold calcium channel and which is characterised in that it consists of at least one β subunit or a fragment of same comprising at least the BID domain, which is fused at the NH₂ or COOH end thereof with the I-II loop of an α₁ subunit or a fragment of same comprising at least the AID domain. The invention also relates to the applications of said protein in the study of cell signalling pathways that are dependent on G-protein-coupled receptors (GPCR) and the identification of compounds that modulate the activity of G proteins.

The present invention relates to a recombinant chimeric protein derived from the α₁ and β subunits of high-threshold calcium channels, and also to its applications for studying G-protein-coupled receptor (GPCR)-dependent cell signaling pathways and identifying compounds that modulate G protein activity.

The GPCR class comprises more than a thousand identified members, encoded by genes representing 2 to 5% of the coding potential of the vertebrate genome (El Far and Betz, Biochem. J., 2002, 365, 329-336); there are 27 genes encoding Gα subunits, 5 encoding the Gβ subunits, and 14 encoding the Gγ subunits (Albert and Robillard, Cell, 2002, 14, 407-418).

Many biological processes, such as synaptic regulation, response to hormones and to pheromones, cell guiding (chemoattraction or chemorepulsion) or vision, involve G-protein-coupled receptors. In fact, GPCRs are capable of providing the recognition and the translation of messages as varied as those of amino acids (glutamic acid, etc.), peptides (angiotensin, neurotensin, somatostatin, etc.), proteins (thyrotropin (TSH), follicle-stimulating hormone (FSH), etc.), amines (acetylcholine, adrenaline, serotonin, etc.), lipids (prostaglandins, leukotrienes, etc.), nucleotides and nucleosides (adenosine or ATP). Ions (Ca⁺⁺), olfactory and taste molecules, photons and pheromones are also part of the extracellular signals recognized by GPCRs (for review, see Gether, Endocrine reviews, 2000, 21, 90-113 and Albert and Robillard, mentioned above).

The extracellular signal is transduced inside the cell by means of heterotrimeric G proteins that bind guanyl nucleotides (GDP and GTP), made up of subunits called Gα, Gβ, Gγ; recognition of the extracellular signal by the GPCR leads to activation of the G proteins, which results in dissociation of the heterotrimer to Gα, and Gβγ, and binding of the Gα subunit to GTP.

Several intracellular effectors can be directly or indirectly modulated through activation of the various Gα and Gβγ subunits of G proteins. The effectors controlled by the Gα subunits may be enzymes (phospholipases A2 and C, adenylyl- and guanylylcyclases, c-jun kinase, tyrosine phosphatase (SH-PTP2), etc.), the activation of which will influence the amount of second messengers produced or released (phosphoinositides and diacyl glycerols, Ca⁺⁺, cAMP, cGMP, etc.), channels (potassium-, calcium-, sodium- or chlorine-conducting), ion exchangers (sodium/proton) or, more recently, kinases (Btk tyrosine kinases (Bruton's tyrosine kinase), MAP kinases (mitogen-activated protein kinase)) (Albert and Robillard, mentioned above).

Gβγ can also modify the activity of at least as many effectors as those controlled by Gα, namely: channels (voltage-dependent sodium-conducting or calcium-conducting channels (N and P/Q) or inward rectifier potassium channels (GIRK: G protein inward rectifier K+ channel), etc.), “conventional” enzymes (phospholipases A2 and C, adenylyl cyclase I, II and IV, tyrosine phosphatase (SH-PTP1), etc.), and also a considerable number of kinases (phosphoinositide 3-kinase, β-adrenergic receptor kinases, c-jun kinase, MAP kinases, Btk tyrosine kinases and T-cell-specific kinase (Tsk) (for review, see Albert and Robillard, mentioned above).

Thus, the study of these signaling pathways and the search for drugs that act on these signaling pathways is of considerable therapeutic interest in the search for novel medicinal products.

It emerges from the above that the activation of G proteins and the dissociation thereof into Gα and Gβγ subunits is the crossroads for a large number of cell signaling and regulatory pathways. Consequently, analysis of the activation of these G proteins makes it possible to study G-protein-coupled receptor-dependent cell signaling pathways and to screen agonists and antagonists of these signaling pathways.

To analyze the activation of G proteins, Janetopoulos et al. (Science, 2001, 291, 2408-2411) have described a technique for the real-time monitoring, in vivo, of the Gα-Gβ interaction. This technique, developed in the amoeba Dictyostelium discoideum, is based on the cotransfection of two constructs encoding two fluorescent chimeric proteins: Gβ-YFP and Gα₂-CFP. The interaction between the two chimeras induces a fluorescence transfer process (FRET), which makes it possible to follow, in real time, their interaction in the living cell. The two chimeras constructed by Janetopoulos et al. are capable of forming a complex that interacts functionally with the cAMP receptor and can be activated by GTPγS. This technique using fluorescence can be adapted to high throughput screening approaches. However, due to the competition with endogenous Gα or Gβγ subunits that suppresses the FRET process, this approach can only function in cells that have had their endogenous equivalent G protein genetically deleted. In addition, in vertebrates, the various isoforms of G proteins are involved in the response to activation of the various GPCR-type receptors. Consequently, the approach of Janetopoulos et al. presumes the construction of a chimera and of a cell line specifically deleted for each isotype of G protein studied.

It emerges from the above that no ubiquitous tool that is simple to use exists for evaluating G protein activation in eukaryotic cells.

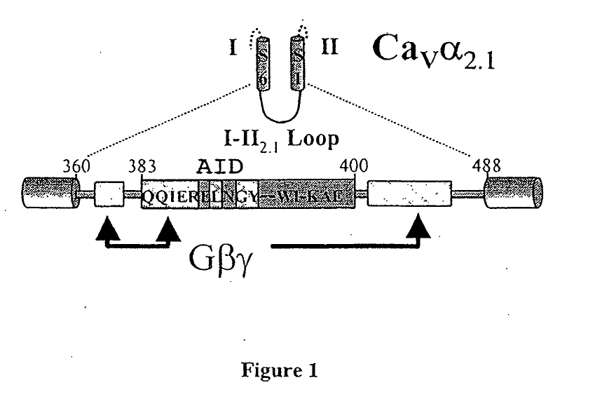

Calcium channels comprise low-threshold channels that are activated by weak depolarizations, and high-threshold channels that are activated by strong depolarizations. The high-threshold channels represent a heteromultimeric complex α₁α₂δβ and γ, in which the membrane-bound α₁ subunit, constituting the channel per se, is associated with an intracellular regulatory β subunit (or Caᵥβ) via its interaction domain (AID domain for alpha interaction domain), having a conserved motif: QQ-E--L-GY--WI---E (one-letter code: - representing any amino acid; Pragnell et al., Nature, 1994, 368, 67-70; FIG. 1) in which residues Y392, W395 and I396 are essential for the biding of the β subunit (De Waard et al., FEBS, 1996, 380, 272-276). The regulatory β subunit binds to the AID domain by its BID domain (beta interaction domain; De Waard et al., J. Biol. Chem., 1995, 270, 12056-12064), which is included in a GK-like domain (Hanlon et al., FEBS, 1999, 445, 366-370).

Seven α₁ subunits have been identified: α_{1A}(Caᵥα_{2.1}), α_{1B} (Caᵥα_{2.2}) α_{1E} (Caᵥα_{2.3}) that form respectively the neuronal channels of P/Q and N type and the channels of R type, regulated by G proteins (G protein-sensitive channels), and α_{1S}(Caᵥα_{1.1}), α_{1c}(Caᵥα_{1.2}), α_{1d}(Caᵥα_{1.3}) and α_{1f}(Caᵥα_{1.4}), that form the G protein-insensitive L-type channels, including the cardiovascular channels (α_{1c}) and skeletal channels (α_{1S}) (Lory et al., m/s, 2001, 10, 979-988).

In the central nervous system, the N- and P/Q-type high-threshold calcium channels are directly involved in the triggering of synaptic function; the opening thereof under the effect of an action potential induces calcium entry into the presynaptic terminal. This signal triggers the secretion of neuromediators such as glutamate into the synaptic cleft, and thus the propagation of the nervous influx in the postsynaptic dendrite. N and P/Q channels are regulated by trimeric G-protein-coupled receptors (GPCRs) such as class III metabotropic glutamate receptors (for review: El Far and Betz, mentioned above) or noradrenergic, muscarinic, GABAergic (GABA: 5-γ-aminobutyric acid), serotoninergic or dopaminergic receptors, and opiate receptors (for review: Hille, Trends NeuroSci., 1994, 17, 531-536). It has been shown that the Gβγ subcomplex is directly responsible for inhibition of the activity of P/Q channels that results from direct binding of Gβγ to the intracytoplasmic loop connecting membrane domains I and II (loop I-II) of the α₁ subunit (De Waard et al., Nature, 1997, 385, 446-450). As a result, this loop has several sites of interaction with Gβγ, that overlap the Caᵥβ regulatory subunit-binding domain (AID domain; FIG. 1) a consensus motif QQ--R-L-GY of which, included in the AID domain, is essential for the binding of Gβγ (FIG. 1; De Waard et al., Nature 1997, 385, 446-450; Zamponi et al., Nature, 1997, 385, 442-446). Furthermore, the Caᵥβ regulatory subunit appears to oppose the functional effect of G proteins (Bourinet et al., P.N.A.S., 1996, 93, 1486-1491). Thus, it would appear that this antagonism involves physical competition between the Caᵥβ subunit and the Gβγ protein at the AID region of the I-II loop (Dolphin et al., J. Physiol., 1998, 506, 3-11).

The inventors have constructed chimeric proteins by NH₂- and/or COOH-terminal fusion:

(i) of the I-II loop of the α₁ subunit of G-protein-sensitive or -insensitive high-threshold calcium channels (respectively, α_{1A} or Caᵥα_{2.1} constituting a P/Q-type neuronal channel, and α_{1c} or Caᵥα_{1.2} constituting an L-type cardiovascular channel) or a fragment thereof; said loop corresponds to positions 367 to 487 with reference to the sequence of the Caᵥα_{2.1} subunit, and comprises the domain for binding to a β subunit of a calcium channel (or AID domain) and the sites for binding to a Gβ subunit of a G protein (FIG. 1), and

(ii) of a β subunit of a high-threshold calcium channel, capable of binding to said fragment of the α₁ subunit.

They have shown that all the chimeric proteins obtained, comprising the I-II loop of an α₁ subunit derived from a G-protein-sensitive or -insensitive calcium channel or a fragment thereof including at least the AID domain, have surprising properties of intramolecular interaction between the binding domains of the α₁ and β subunits of the calcium channel, that prevent the binding of the chimera to the AID domain of an α₁ subunit. They have confirmed that this masking of the binding domain of the β subunit was indeed due to its intramolecular interaction with the AID domain, since deletion of the AID domain from the chimera re-establishes this binding. They have also shown that the binding of the chimera comprising the I-II loop of a “G-protein-sensitive” α_{1A} subunit, to the α₁ subunit interaction domain is re-established by the addition of Gβγ. This result was confirmed by the demonstration, ex vivo, of the interaction of a recombinant β subunit labeled with a Cy3-type fluorophore, with a fluorescent chimera of the α_{1A} subunit (GFP-α_{1A}), by means of fluorescence transfer (FRET) measurement using confocal microscopy. These properties have allowed them to demonstrate that, unexpectedly, the regulation of P/Q channels involves a displacement, by the Gβγ complex, of the interaction between the regulatory β subunit and the α subunit of the calcium channel, and not its inhibition, as had been previously suggested.

More precisely, the inventors have shown that the chimeric protein derived from a G-protein-sensitive α₁ subunit exists in two forms, that are “closed” or “open”, respectively in the absence or in the presence of a Gβ subunit capable of binding to said fragment of the a₁ subunit, either in the form of a Gβ monomer or in the form of a Gβγ heterodimer. In the absence of Gβ (or Gβγ), the chimeric protein is capable of folding on itself, thus allowing the interaction domains of the α₁ and β subunits of the calcium channel to associate by means of a stable intramolecular binding (closed form). In the presence of Gβ (or Gβγ), the intramolecular binding is destroyed and the interaction domains of the α₁ and β subunits of the calcium channel dissociate (open form), thus allowing each of the domains to interact, respectively, with Gβ (α₁ subunit interaction domain: AID domain) and/or an α₁ subunit of a calcium channel (β subunit interaction domain: BID domain).

Similarly, the chimeric protein derived from a G-protein-insensitive α₁ subunit is capable of folding on itself, thus allowing the interaction domains of the α₁ and β subunits of the calcium channel to associate by means of a stable intramolecular binding (closed form). Consequently, in the presence of antagonists for this binding, other than Gβ or Gβγ, the intramolecular binding can also be destroyed and the interaction domains of the α₁ and β subunits of the calcium channel dissociate (open form), thus allowing each of the domains to interact, respectively, with said antagonist other than Gβ or Gβγ (α₁ subunit interaction domain: AID domain) and/or an α₁ subunit of a calcium channel (β subunit interaction domain: BID domain).

Consequently, due to the reorganization of their structure in the presence of free Gβ or Gβγ subunits or else other antagonists of the interaction between the α₁ and β subunits (change from the closed form to the open form), the chimeric proteins derived from the α₁ and β subunits of high-threshold calcium channels represent sensitive, specific tools that are simple to use, and are useful for the following applications:

the chimeric proteins derived from a G-protein-sensitive α₁ subunit (for example: α_{1A}, α_{1B} and α_{1E}) make it possible to determine the variations in cellular concentration of free Gβγ subunits, ex vivo, in real time and therefore to measure the activation of G proteins in cells: such chimeric proteins represent ubiquitous biosensors for G protein activation that are entirely suitable for studying G-protein-coupled receptor-dependent cell signaling and regulatory pathways and for screening agonists/antagonists of these signaling pathways that are capable of increasing or of decreasing the concentration of free Gβγ subunits in cells and therefore of modulating the activity of these G-protein-coupled receptor-dependent cell signaling and regulatory pathways;

the chimeric proteins derived from a G-protein-sensitive or -resistant α₁ subunit (for example: α_{1A}, α_{1B}, α_{1E}, α_{1c}, α_{1d}, α_{1S} amd α_{1f}) represent simple, sensitive and specific tools that are entirely suitable for screening antagonists of the interaction between the α₁ and β subunits, that are capable of modulating the activity of all high-threshold.calcium channels;

the chimeric proteins derived from an α₁ subunit and from a β subunit of a high-threshold calcium channel, as defined above, are also useful for the systematic pharmacotoxicological control of new medicinal products in phase I and the search for natural agonists of orphan receptors. In fact, the cloning of the human genome has made it possible to identify approximately 350 GPCR receptors, Among these, only 200 have an identified ligand.

The others, called orphan receptors, potentially constitute key targets for the identification of novel cell signaling and regulatory pathways. The search for agonists and for antagonists of these receptors is therefore of major interest both from the point of view of fundamental research and from a therapeutic point of view.

Consequently, a subject of the present invention is a chimeric protein derived from a high-threshold calcium channel, characterized in that it comprises at least one β subunit or a fragment thereof including at least the BID domain, fused, at its NH₂ or COOH end, with the I-II loop of an α₁ subunit or a fragment thereof including at least the AID domain.

In accordance with the invention, the AID and BID domains are as defined above; the I-II loop of the α₁ subunit comprises the AID domain for binding to the β subunit and the sites for binding to the Gβ subunit of a G protein, including a consensus binding site that is included in this AID domain. These various domains are illustrated in FIG. 1.

The invention encompasses the chimeric proteins derived from the α₁ and β subunits of vertebrates, in particular of human or nonhuman mammals and of their orthologs in invertebrates.

Chimeric proteins in accordance with the invention are represented in particular by:

a β subunit fused, at its NH₂ or COOH end, with the I-II loop of an α₁ subunit, and

the GK-like domain of a β subunit including the BID domain (Hanlon et al., mentioned above), fused, at its NH₂ or COOH end, with the I-II loop of an α₁ subunit.

In accordance with the invention, the I-II loop, or a fragment thereof, is either fused directly to the NH₂ or COOH end of the β subunit or of a fragment thereof, or the two sequences are separated by means of a spacer peptide whose size and amino acid sequence are such that the AID and BID domains of the chimeric protein containing said spacer are capable of interacting so as to form intramolecular binding that is displaced in the presence of an antagonist (change from the closed form to the open form); such a spacer peptide is in particular represented by a polyglycine sequence.

According to an advantageous embodiment of said chimeric protein, it is derived from a G-protein-sensitive high-threshold calcium channel.

According to an advantageous provision of this embodiment, said chimeric protein comprises a fragment of an α₁ subunit selected from α_{1A}, α_{1B} and α_{1E}.

According to another advantageous embodiment of said chimeric protein, said β subunit is selected from the group consisting of β₁, β₂, β₃ and β₄.

The invention also encompasses the chimeric proteins consisting of sequences that are functionally equivalent to the sequences as defined above, i.e. in which the β subunit and the I-II loop of the α₁ subunit or fragments thereof as defined above are capable of forming intramolecular binding by means of their interaction domains; said binding being optionally destroyed in the presence of free Gβ or Gβγ subunits or else other antagonists of the interaction between the α₁ and β subunits (“open form”).

Among these sequences, mention may, for example, be made of the sequences derived from the preceding sequences by:

mutation (substitution and/or deletion and/or addition) of one or more amino acids of the sequences as defined above,

modification of at least one —CO—NH— peptide bond of the peptide chain of the chimeric protein as defined above, in particular by replacement with a bond different from the —CO—NH— bond (methyleneamino, carba, ketomethylene, thioamide, etc.) or by introduction of a retro-type or retro-inverso-type bond, and/or

substitution of at least one amino acid of the peptide chain of the chimeric protein as defined above, with a nonproteinogenic amino acid residue.

The term “nonproteinogenic amino acid residue” is intended to mean any amino acid that is not part of the constitution of a natural protein or peptide, in particular any amino acid in which the carbon bearing the side chain R, namely the —CHR— group, located between —CO— and —NH— in the natural peptide chain, is replaced with a motif that is not part of the constitution of a natural protein or peptide.

A subject of the present invention is in particular a variant chimeric protein derived from a chimeric protein as defined above, characterized in that it has a mutation of at least one amino acid in the sequences of said β subunit and/or of the I-II loop of an α₁ subunit.

According to another advantageous embodiment of said chimeric protein, said variant has a mutation that modifies the affinity of the β subunit for the I-II loop of the α₁ subunit and/or vice versa; such mutations make it possible to obtain a chimeric protein that is more or less sensitive to the concentration of free Gβ or Gβγ subunits.

Among these mutations, mention may be made of mutations of the AID domain of the I-II loop of the α₁ subunit, as described in Pragnell et al., mentioned above, and De Waard et al., FEBS, 1996, 380, 272-276, i.e.: Q383A, Q384A, E386D, E386S, L389H, G391R, Y392S, Y392F, W395A, I396A and E400A.

According to another advantageous embodiment of said chimeric protein or of its variant, it is coupled, preferably covalently, to at least one suitable label allowing the detection and/or the purification and/or the immobilization of said protein, for example: an antigenic epitope, a polyhistidine-type tag, or a luminescent compound (fluorophore such as GFP or one of its variants: CFP, YFP and BFP), a radioactive compound or an enzymatic compound.

In accordance with the invention, said coupling is carried out by any appropriate means, in particular via a peptide bond by means of the COOH- and/or NH₂-terminal functions of the peptide chain, or else via another covalent bond, for instance: an ester, ether, thioether or thioester bond, by means of reactive functions of the side chain of an amino acid of the peptide chain.

According to an advantageous provision of this embodiment, said chimeric protein comprises an acceptor or donor fluorophore respectively at its NH₂ and/or COOH end.

The acceptor fluorophores, for example CFP or BFP, can be coupled without distinction at the NH₂ or COOH end of the chimeric protein, the donor fluorophores, for example CFP or YFP, are fused to the opposite end of said chimeric protein. Such chimeric proteins are useful for the real-time ex vivo study of G protein activation and the screening of molecules capable of modulating this activation, by measurement of fluorescence transfer (FRET).

In fact, the labeling with a luminescent compound has the advantage of obtaining a localized signal that does not require the presence of other reagents, as is the case for enzymatic labelings. This type of labeling also makes it possible to use a phenomenon such as energy transfer that can take place according to various mechanisms: resonance energy transfer, radiative energy transfer (the acceptor absorbs the light emitted by the donor) and electron transfer.

This energy transfer, between a luminescent “donor” compound (D) and a luminescent or nonluminescent “acceptor” compound (A), and which depends on the distance between A and D, has been used for carrying out many assays. D and A, which are coupled to each end of the chimeric protein so that the energy transfer takes place only when the intramolecular interaction between the BID and AID domains takes place (closed form), are chosen. This phenomenon results in a decrease or quenching of the luminescence of D and an emission of luminescence from A if the latter is luminescent, when D is excited. During these assays, either the variation in luminescence of A or the variation in luminescence of D is measured, the nature of A and of D being variable. For example, to measure the variation in luminescence of A, two fluorescent proteins can be used as donor and acceptor, or else a complex of rare earth metals (europium, terbium) with a chelate, a cryptate or a macrocycle can be used as donor and a fluorescent protein can be used as acceptor. The measurement of the variation in luminescence of D is based on the ability of a compound (A) to decrease or eliminate the luminescence of another compound (D) when they are sufficiently close (“quench”). The range of molecules A that can be used is therefore broader and thus includes nonluminescent compounds such as heavy metal, heavy atoms, chemical molecules, for instance methyl red, nanoparticles such as those sold under the name Nanogold® by the company Nanoprobes (USA), or else the molecules sold under the names DABCYL® (Eurogentec, Belgium), QSY Dyes (Molecular Probes Inc., USA), ElleQuencher® (Oswell/Eurogentec) or Black Hole Quenchers® (Biosearch Technologies Inc., USA).

A subject of the present invention is also a peptide, characterized in that it comprises a fragment of at least 7 amino acids of the sequence of the chimeric protein as defined above, located at the junction of the β subunit and of the I-II loop of the α₁ subunit or of their fragments as defined above; such peptides make it possible in particular to produce antibodies specific for the chimeric protein according to the invention.

A subject of the present invention is also antibodies, characterized in that they are directed against a chimeric protein or a peptide as defined above.

In accordance with the invention, said antibodies are either monoclonal antibodies or polyclonal antibodies.

These antibodies can be obtained by conventional methods, that are known in themselves, comprising in particular the immunization of an animal with a protein or a peptide in accordance with the invention, in order to make it produce antibodies directed against said protein or said peptide.

Such antibodies are useful in particular for immobilizing the chimeric protein on a solid support, purifying it, or else detecting it.

A subject of the present invention is also a nucleic acid molecule, characterized in that it is selected from the group consisting of the sequences encoding a chimeric protein or a peptide as defined above and the sequences complementary to the above sequences, that may be sense or antisense.

A subject of the invention is also probes and primers, characterized in that they comprise a sequence of approximately 10 to 30 nucleotides corresponding to that located at the junction of the β subunit and of the I-II loop of the α₁ subunit or of their fragments as defined above; these probes and these primers make it possible to specifically detect/amplify said nucleic acid molecules encoding the chimeric protein according to the invention.

A subject of the invention is also other primers for specifically amplifying the β subunit and/or the I-II loop of the α₁ subunit or their fragments as defined above, characterized in that they are selected from the group consisting of the sequences SEQ ID NO: 1, 2, 4, 6, 7, 8 and 9.

The nucleic acid molecules according to the invention are obtained by conventional methods, that are known in themselves, according to standard protocols such as those described in Current Protocols in Molecular Biology (Frederick M. AUSUBEL, 2000, Wiley and son Inc, Library of Congress, USA).

The sequences encoding a chimeric protein according to the invention can be obtained by amplification of a nucleic acid sequence by PCR or RT-PCR using a suitable pair of primers, or else by screening genomic DNA libraries by hybridization with a homologous probe.

The derived nucleic acid molecules, encoding a variant of the chimeric protein according to the invention, are obtained by conventional methods for introducing mutations into a nucleic acid sequence, that are known in themselves, according to the abovementioned standard protocols. For example, the sequence encoding a variant of the chimeric protein according to the invention can be obtained by site-directed mutagenesis according to the method of Kunkel et al. (P.N.A.S., 1985, 82, 488-492).

A subject of the present invention is also a recombinant eukaryotic or prokaryotic vector, characterized in that it comprises an insert consisting of the nucleic acid molecules encoding a chimeric protein as defined above.

Preferably said recombinant vector is an expression vector in which said nucleic acid molecule or one of its fragments is placed under the control of suitable regulatory elements for transcription and translation. In addition, said vector can comprise sequences fused in frame with the 5′ and/or 3′ end of said insert, that are useful for immobilizing and/or detecting and/or purifying the protein expressed from said vector. Many vectors into which it is possible to insert a nucleic acid molecule of interest in order to introduce it into and to maintain it in a eukaryotic or prokaryotic host cell are known in themselves; the choice of a suitable vector depends on the use envisioned for this vector (for example, replication of the sequence of interest, expression of this sequence, maintenance of the sequence in extrachromosomal form or else integration into the host's chromosomal material), and also on the nature of the host cell. For example, viral or nonviral vectors such as plasmids can be used.

These vectors are constructed and introduced into host cells by conventional recombinant DNA and genetic engineering methods that are known in themselves.

According to one embodiment of said recombinant vector, it is a eukaryotic expression vector having a sequence selected from the group consisting of the sequences SEQ ID NO: 5 and SEQ ID NO: 10; the plasmid SEQ ID NO: 5 contains the I-II loop of the rabbit Caᵥα_{2.1} subunit, fused to the C-terminal end of a rat Caᵥβ₃ subunit, under the control of the CMV promoter, and the plasmid SEQ ID NO: 10 contains an insert consisting, from 5′ to 3′, of the in-frame fusion of the following fragments: the sequence GAP-43, the cDNA encoding EGFP (fluorescence donor), the GK-like domain of the rat Caᵥβ₃ subunit, the I-II loop of the rabbit Caᵥα_{2.1} subunit and the cDNA encoding CFP (fluorescence acceptor).

A subject of the present invention is also cells modified with a chimeric protein, a nucleic acid molecule or else a recombinant vector as defined above.

According to an advantageous embodiment of the invention, said cells are eukaryotic cells.

According to an advantageous provision of this embodiment, said cells express at least one G-protein-coupling receptor (GPCR); said cells are either cells constitutively expressing at least one GPCR, or modified cells that express a recombinant GPCR.

Modified cells in accordance with the invention can be obtained by any means, that are known in themselves, for introducing a nucleic acid molecule or a protein into a host cell. For example, in the case of animal cells, use may be made, inter alia, of viral vectors such as adenoviruses, retroviruses, lentiviruses and AAVs, into which the sequence of interest has been inserted beforehand; said nucleotide sequence (isolated or inserted into a plasmid vector) or peptide sequence can also be combined with a substance that allows it to cross the host-cell membrane, for example a preparation of liposomes, of lipids or of cationic polymers, or else it can be injected directly into the host cell.

A subject of the present invention is nonhuman transgenic animals and in particular mammals, characterized in that all or some of their cells are transformed with a nucleic acid molecule according to the invention. They are, for example, animals into which a sequence encoding the chimeric protein according to the invention, under the control of suitable regulatory elements for transcription and translation, has been introduced. Such transgenic animals are useful in particular for the secondary screening steps: i) for evaluating the cell, or even tissue, targeting of a molecule active on GPCRs or calcium channels, that was identified in a primary screen, ii) for studying the bioavailability of such a molecule, and iii) for investigating, in a first approach, possible side effects of such a molecule.

The subject of the present invention is also the use of a product selected from the group consisting of the chimeric proteins, the nucleic acid molecules, the recombinant vectors, the modified cells and the nonhuman transgenic mammals as defined above, for studying G-protein-coupled receptor-dependent cell signaling and regulatory pathways.

A subject of the present invention is also the use of a product selected from the group consisting of the chimeric proteins, the nucleic acid molecules, the recombinant vectors, the modified cells and the nonhuman transgenic mammals as defined above, for screening agonists and/or antagonists of G-protein-coupled receptor-dependent cell signaling and regulatory pathways.

A subject of the present invention is also the use of a product selected from the group consisting of the chimeric proteins, the nucleic acid molecules, the recombinant vectors, the modified cells and the nonhuman transgenic mammals as defined above, for screening antagonists of the interaction between the α₁ and β subunits of high-threshold calcium channels; such antagonists are useful for modulating the activity of all high-threshold calcium channels and therefore represent medicinal products that can be used in the treatment of diseases associated with a dysfunction of calcium homeostasis and of pathologies where modulation of calcium entry can compensate for a cellular deficiency, in particular epilepsy, ataxia, migraines, hypo- and hypercalcemia in the muscles, diabetes, and cardiovascular diseases.

According to an advantageous embodiment of the invention, the study of the G-protein-coupled receptor-dependent cell signaling and regulatory pathways is carried out by means of a method comprising at least the following steps:

a₁) culturing of modified cells expressing a chimeric protein derived from a G-protein-sensitive calcium channel and a G-protein-coupled receptor, as defined above,

b₁) transduction of a signal via said G-protein-coupled receptor, by any appropriate means, and

c₁) determination, by any appropriate means, of the proportion of said chimeric protein expressed in said cells that is bound to a Gβγ subunit.

Such a determination makes it possible to evaluate the variations in cellular concentration of free Gβγ subunits and therefore to measure the G protein activation in the cells.

According to an advantageous embodiment of the invention, the screening of agonists/of antagonists of the G-protein-coupled receptor-dependent cell signaling and regulatory pathways is carried out by means of a method comprising at least the following steps:

a₂) culturing of modified cells expressing a chimeric protein derived from a G-protein-sensitive calcium channel and a G-protein-coupled receptor, as defined above,

b₂) transduction of a signal via said G-protein-coupled receptor, by any appropriate means,

c₂) comparative determination, by any appropriate means, of the proportion of said chimeric protein expressed in the cells that is bound to a Gβγ subunit, before and after the bringing into contact of said cells in b₂) with a molecule to be tested, and

d₂) identification of the molecules that are agonists/antagonists of the G-protein-coupled receptor-dependent cell signaling and regulatory pathways, corresponding to those capable respectively of increasing and of decreasing the cellular concentration of free Gβγ subunits.

Advantageously, said modified cells in a₁) or in a₂) express a chimeric protein as defined above coupled, at its NH₂ and COOH ends, respectively to a fluorescence donor fluorophore and a fluorescence acceptor fluorophore, and said determination in c₁) or in c₂) is carried out by means of the fluorescence transfer (FRET) technique.

According to an advantageous embodiment of the invention, the screening of antagonists of the interaction between the α₁ and β subunits of high-threshold calcium channels is carried out by means of a method comprising at least the following steps:

a₃) bringing a molecule to be tested into contact with a chimeric protein derived from a G-protein-sensitive or -insensitive calcium channel as defined above and with a peptide comprising the AID domain of a G-protein-insensitive α₁ subunit,

b₃) measuring, by any appropriate means, the binding of said chimeric protein to said peptide, and

c₃) identifying the antagonists of the interaction between the α₁ and β subunits corresponding to those with which binding of said chimeric protein to said peptide is observed.

According to an advantageous embodiment of said method, said peptide comprising the AID domain is immobilized on a solid support, and said chimeric protein is coupled to a label for measuring said binding in b₃), as defined above, in particular a fluorophore.

A subject of the invention is also a kit for implementing the methods as defined above, characterized in that it includes at least one of the following products: a chimeric protein, an antibody, a recombinant vector, a modified cell or a nonhuman transgenic mammal, as defined above.

The chimeric protein of the invention has the following advantages:

it constitutes a ubiquitous biosensor for endogenous free Gβγ subunits that is suitable for the real-time study of G-protein-coupled receptor-dependent cell signaling and regulatory pathways, and for the systematic screening (high-throughput screening) of molecules capable of modulating them, that can potentially be used as a medicinal product for the treatment of diseases in which a dysfunction of these pathways is observed, in particular immune system pathologies (for review, see Lombardi et al., Crit. Rev. Immunology, 2002, 22, 141-163; Onuffer and Horuk, Trends in Pharmacol, 2002, 23, 459-467) and neuropsychiatric and cardiovascular pathologies (Seifert and Wenzel-Seifert, Naumyn-Schmeideberg's Arch. Pharmacol., 2002, 366, 381-416). In addition, its use is simple insofar as it makes it possible to partially do away with problems of stoichiometry since its use involves only two molecules (Caᵥβ/Caᵥα-Gβγ) instead of three partners (Caᵥα/Caᵥβ/Gβγ) for the methods of the prior art;

it is suitable for systematic screening (high-throughput screening) of molecules capable of modulating the activity of high-threshold calcium channels, that can potentially be used as a medicinal product for the treatment of diseases in which a dysfunction of calcium homeostasis is observed and of pathologies where the modulation of calcium entry can compensate for a cellular deficiency, as defined above.

Besides the above provisions, the invention also comprises other provisions which will emerge from the following description, which refers to examples of use of the chimeric protein that is the subject of the present invention, and also to the attached drawings, in which:

FIG. 1 illustrates the overlap, in the I-II loop of the Caᵥα_{2.1} subunit, of the domains for binding to the β subunit (AID domain) and to the Gβγ complex. The AID domain is represented by a black box (positions 383 to 400). The binding sites for the Gβ (Gβγ) subunit are represented by hatched boxes; the site in the central position (QQ--R-L-GY) that is essential for the binding of the Gβ (Gβγ) subunit is included in the AID domain,

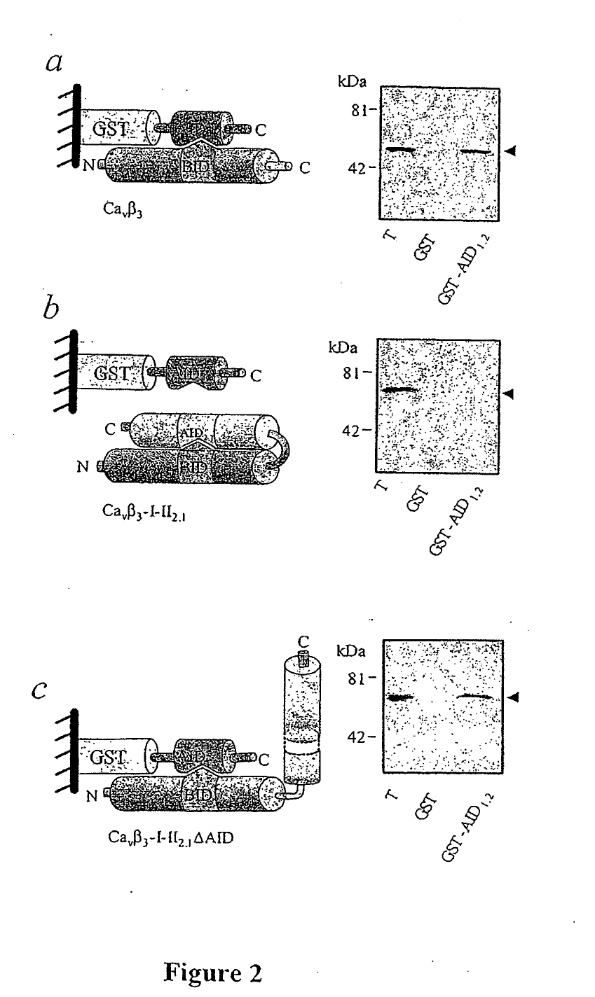

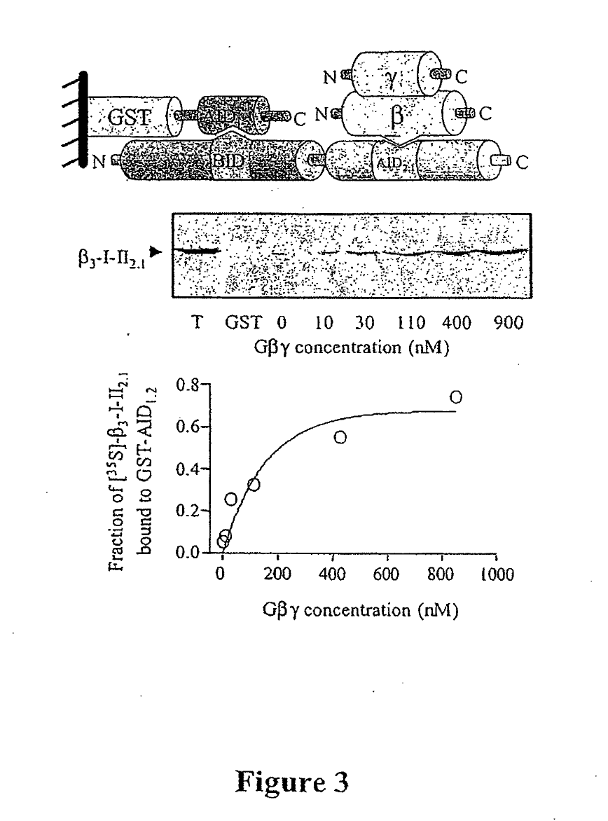

FIGS. 2 and 3 illustrate the displacement of the Caᵥα_{2.1}-Caᵥβ interaction by the G-protein Gβγ complex:

FIG. 2a illustrates the binding of the β3 subunit (1 to 3 pM) with the AID_{1.2} domain of the GST-AID_{1.2} fusion protein (1 μM),

FIG. 2b shows that the fusion of the β3 subunit with the I-II loop of the α2.1 subunit (Caᵥβ₃-I-II_{2.1} chimera) prevents its binding with the AID_{1.2} domain of the GST-AID_{1.2} fusion protein,

FIG. 2c shows that the deletion of the 18 amino acids of the AID_{2.1} domain (Caᵥβ₃-I-II_{2.1}ΔAID chimera) restores the binding of the β3 subunit with the AID_{1.2} domain of the GST-AID_{1.2} fusion protein,

FIG. 3 shows that the addition of Gβγ complex displaces the intramolecular interaction between the Caᵥβ subunit and the I-II loop of the α_{2.1} subunit of the Caᵥβ₃-I-II_{2.1} chimera, thus allowing the β3 subunit to bind with the AID_{1.2} domain of the GST-AID_{1.2} fusion protein; the concentration of Gβγ capable of displacing 50% of the binding between the Caᵥβ subunit and the AID_{2.1} domain (IC₅₀) is 160 nM,

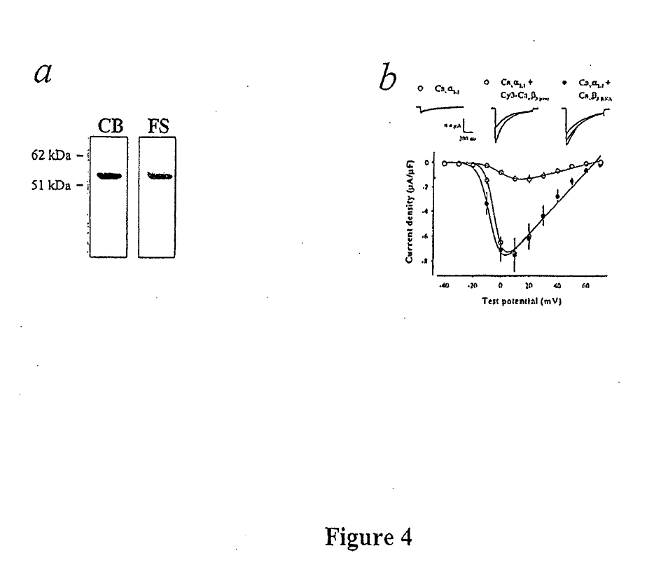

FIGS. 4 to 7 illustrate the FRET analysis of the disassembly of the P/Q calcium channel, induced by the Gβγ complex:

FIG. 4a illustrates the Cy3-labeling of the purified His-Caᵥβ₃ subunit. CB: Coomassie blue staining of an SDS-PAGE gel illustrating the purity of the protein. FS=recording of the fluorescence of an unstained gel showing the covalent labeling of the protein,

FIG. 4b illustrates the effect of the Caᵥβ₃ subunit coupled to a fluorochrome (Cy3-Caᵥβ₃) on the current-voltage relationship of Caᵥα_{2.1} channels expressed in xenopus oocytes, by comparison with the unlabeled Caᵥβ₃ subunit (injection of cRNA),

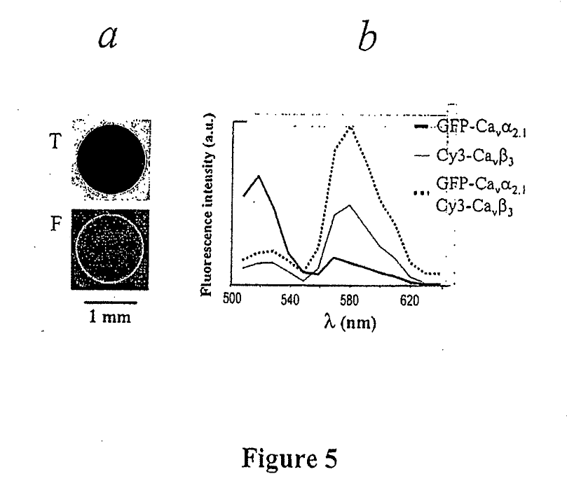

FIG. 5a illustrates the observation by confocal microscopy of two distinct regions of xenopus oocytes containing Caᵥα_{2.1} and Cy3-Caᵥβ₃. T=transmission, F=fluorescence,

FIG. 5b illustrates the fluorescence emission spectrum for GFP-Caᵥα_{2.1}, Cy3-Caᵥβ₃ and (GFP-Caᵥα_{2.1}+Cy3-Caᵥβ₃ ),

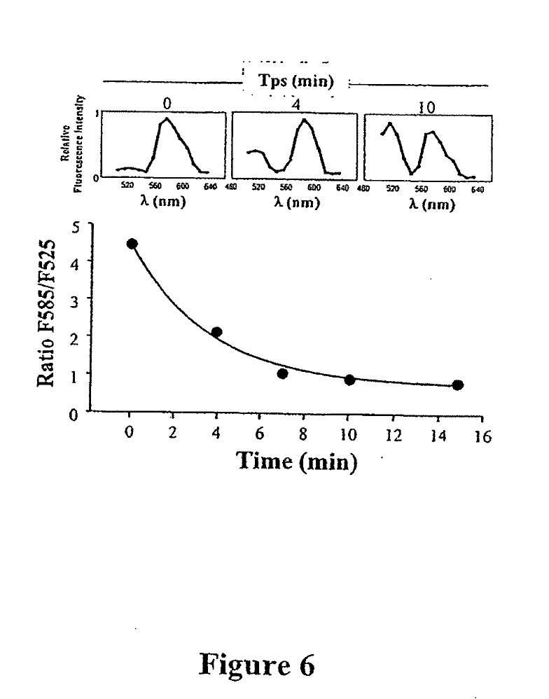

FIG. 6 illustrates the kinetics of decrease in fluorescence transfer induced by the injection of 100 nM of Gβγ. Upper panel: variations in the fluorescence emission spectrum, and lower panel:

variations in the ratio of fluorescence intensities (R_{f}) at 585 nm and 525 nm,

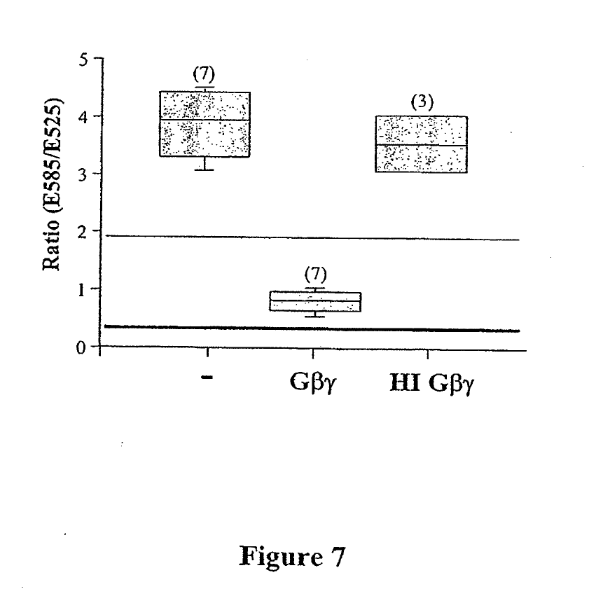

FIG. 7 illustrates the Rf values of the noninjected oocytes (-), oocytes injected with Gβγ (100 nM) or oocytes injected with heat-inactivated Gβγ (HI-Gβγ),

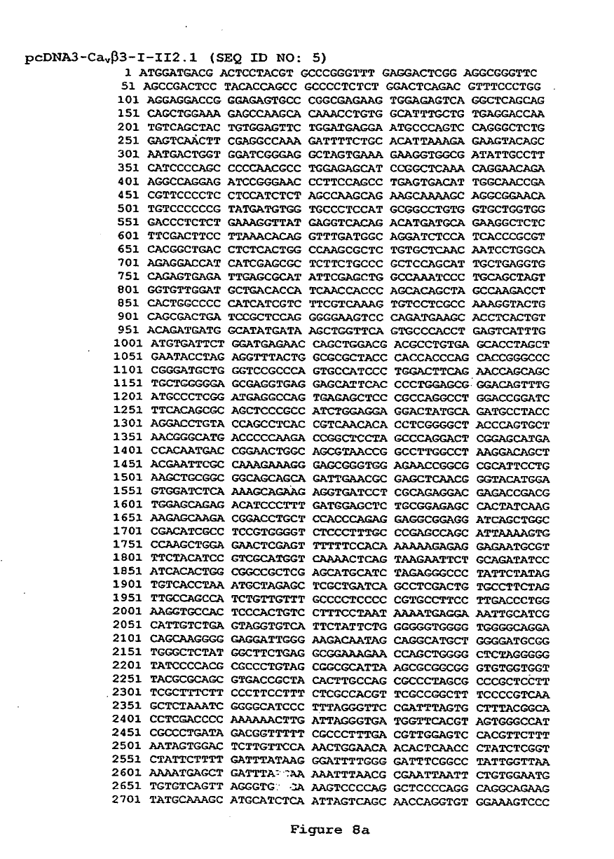

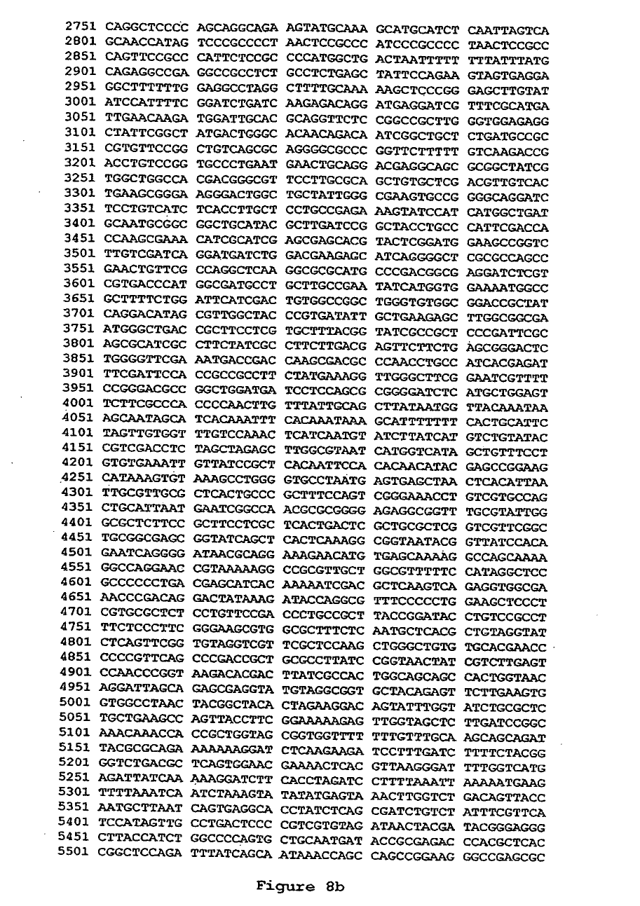

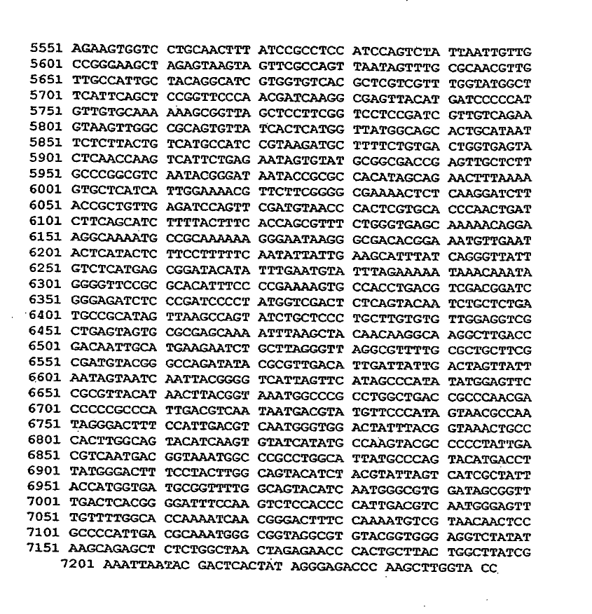

FIG. 8 (a to c) illustrates the sequence of the plasmid pcDNA3Cavβ3-I-II_{2.1} (SEQ ID NO: 5) containing the I-II loop of the rabbit Cαᵥα_{2.1} subunit fused to the C-terminal end of the rat Caᵥβ₃ subunit, under the control of the CMV promoter,

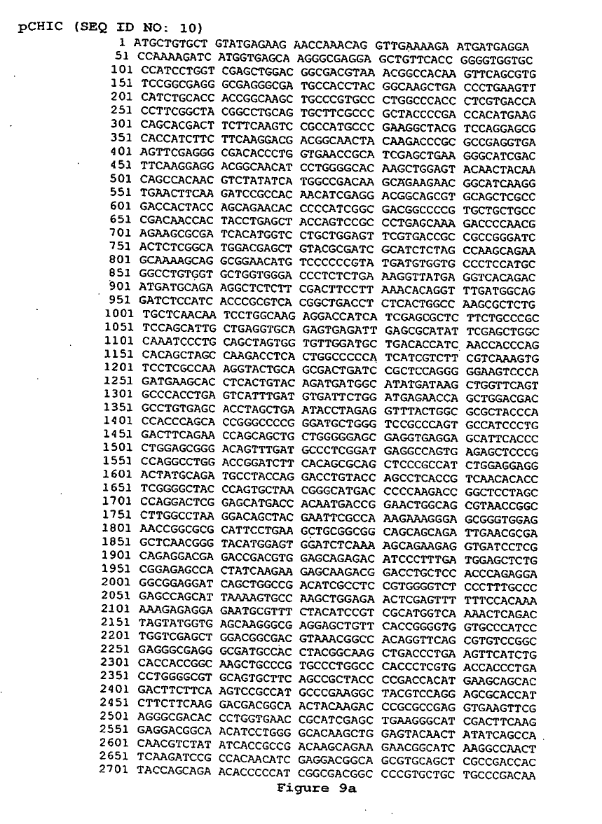

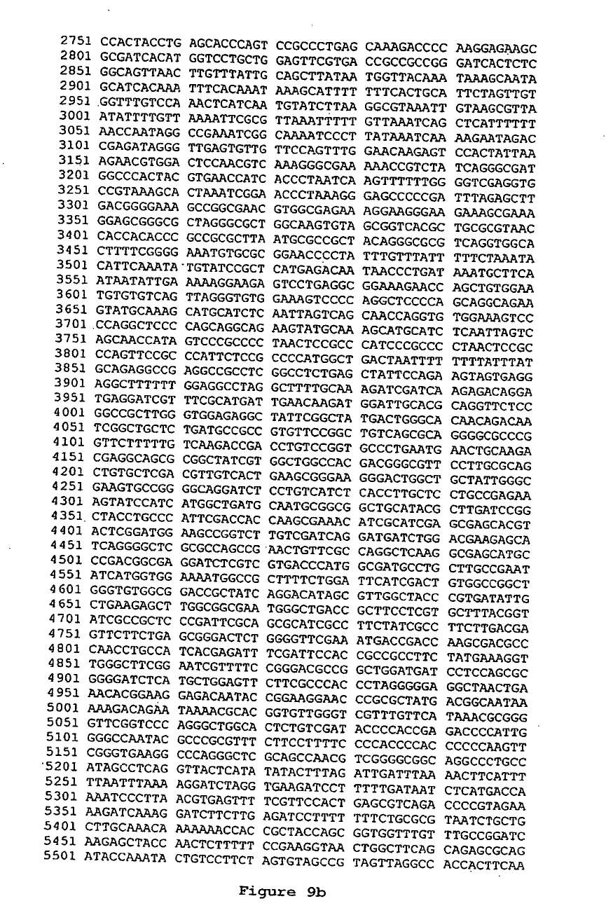

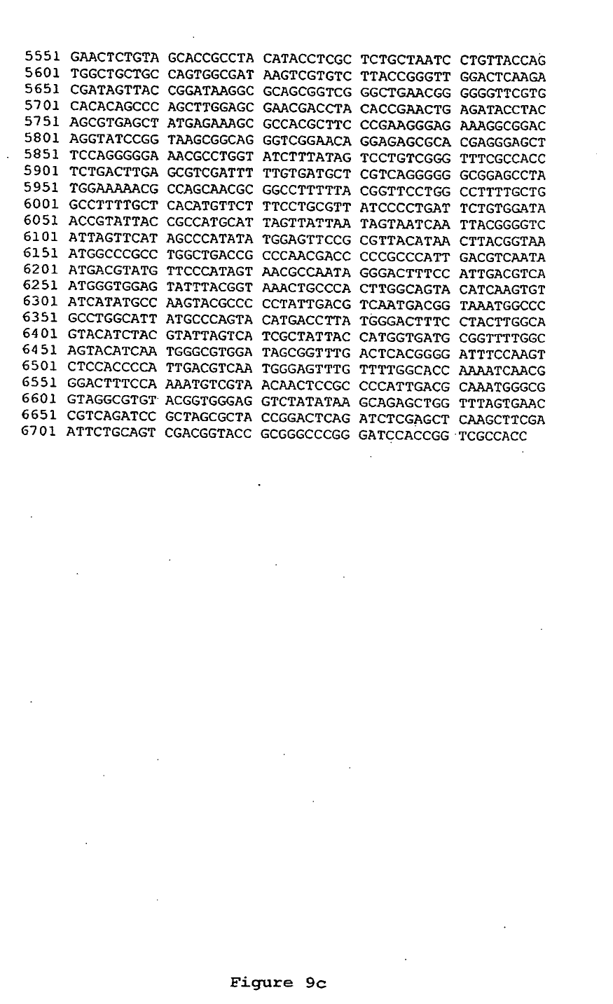

FIG. 9 (a to c) illustrates the sequence of the plasmid pCHIC (SEQ ID NO: 10) derived from the vector pEYFPmemb. (CLONTECH), containing an insert consisting, from 540 to 3′, of the in-frame fusion of the following fragments: the GAP-43 sequence, the cDNA encoding EGFP (fluorescence donor), the GK-like domain of the rat Caᵥβ₃ subunit, the I-II loop of the rabbit Caᵥα_{2.1} subunit and the cDNA encoding CFP (fluorescence acceptor).

It should be clearly understood, however, that these examples are given only by way of illustration of the subject of the invention, of which they in no way constitute a limitation.

### EXAMPLE 1

### Construction of a Recombinant Chimeric Protein Cavβ3-I-II_{2.1}

1) Materials and Methods

The PCR amplification and the cloning of the recombinant DNA are carried out by conventional techniques known to those skilled in the art, according to standard protocols such as those described, for example, in Current Protocols in Molecular Biology (Frederick M. AUSUBEL, 2000, Wiley and son Inc, Library of Congress, USA).

An expression plasmid containing a cDNA encoding a chimeric protein according to the invention, consisting of the C-terminal fusion of the rat β3 subunit with the I-II intracellular loop of the rabbit α₁ subunit, was constructed in the following way:

The cDNA of the rat Caᵥβ₃ subunit (corresponding to positions 98 to 1545 of the Genbank sequence M88755) is amplified by PCR using the following sense and antisense primers:

(SEQ ID NO: 1)

5′-TTTGGTACCATGGATGACGACTCCTACGTGCCCGGGTTTGAGGACTC
GGAGGCGGGTT-3′,
and

(SEQ ID NO: 2)

5′-GCGGAATTCGTAGCTGTCCTTAGGCCAAGGCCGGTTACGCTGCCAGT
T-3′,.

The fragment thus obtained was cloned between the Kpn I and EcoR I sites of the expression plasmid (pcDNA3, Invitrogen), to give the recombinant plasmid pcDNA3-Caᵥβ3.

The cDNA fragment corresponding to the I-II loop of the rabbit Caᵥα_{2.1} subunit (positions 1383 to 1754 of the Genbank sequence X57477), the sequence of which is illustrated in FIG. 1, was amplified by PCR using the following sense and antisense primers: -5′-GGGGAATTCGCCAAAGAAAGGGAGCGGGTGGAGAAC-3′ (SEQ ID NO: 3; De Waard et al., mentioned above and Bichet et al., Neuron, 2001, 25, 177-190), and -5′-TTTGAATTCTTACTGAGTTTTGACCATGCGACGGATGTAGAAACGCATTCT-3′ (SEQ ID NO: 4).

The fragment obtained was cloned into the EcoR I site of the plasmid pcDNA3-Cavβ3, to give the recombinant plasmid pcDNA3-Caᵥβ3-I-II_{2.1}.

A control plasmid containing a cDNA encoding a chimeric protein consisting of the C-terminal fusion of the rat β₃ subunit with the I-II intracellular loop of the rabbit Caᵥα_{2.1} subunit from which the AID domain had been deleted was constructed in a similar manner; the recombinant plasmid thus obtained was called pcDNA3-Cavβ₃-I-II_{2.1}ΔAID.

2) Results

The recombinant plasmid pcDNA3-Caᵥβ3-I-II_{2.1} has the sequence SEQ ID NO: 5. The peptide sequence deduced from the nucleotide sequence obtained by automatic sequencing of the insert cloned into the plasmid pcDNA3-Caᵥβ3-I-II_{2.1} has the sequence expected for a chimeric protein according to the invention. Similarly, the peptide sequence deduced from the nucleotide sequence obtained by automatic sequencing of the insert cloned into the plasmid pcDNA3-Caᵥβ3-I-II_{2.1}ΔAID corresponds to that expected for a chimeric protein from which the AID domain has been deleted.

### EXAMPLE 2

### In Vitro Demonstration of the Displacement of the Caᵥα_{2.1}-Caᵥβ Interaction by the G-Protein Gβγ Complex

1) Materials and Methods

The expression of the recombinant DNA and the analysis of the recombinant proteins are carried out by conventional techniques known to those skilled in the art, according to standard protocols such as those described, for example, in Current Protocols in Molecular Biology (Frederick M. AUSUBEL, 2000, Wiley and son Inc, Library of Congress, USA) and in Current protocols in Immunology (John E. Coligan, 2000, Wiley and son Inc, Library of Congress, USA).

a) Expression of the Recombinant Chimeric Proteins and of the GST-AID_{1.2} Fusion

The chimeric proteins Caᵥβ3-I-II_{2.1} and Caᵥβ3-I-II_{2.1}ΔAID, and Caᵥβ₃ subunit, are transcribed and translated in vitro, in the presence of [³⁵S]-methionine, from the plasmids as described in Example 1, using the Promega TNT system kit according to the supplier's instructions.

The GST-AID_{1.2} fusion protein described in Pragnell et al., mentioned above, is produced and purified as described by the above authors. The GST protein produced and purified under the same conditions is used as a control.

b) In Vitro Analysis of the Regulation of the Caᵥα_{2.1}-Caᵥβ Interaction by the G-Protein Gβγ Complex

The in vitro analysis of the regulation of the Caᵥα_{2.1}-Caᵥβ interaction by the G-protein Gβγ complex is carried out according to the protocols as described in De Waard et al., Nature, 1997, 385, 446-450. More precisely, the labeled β₀₃ subunit and the labeled chimeric proteins ([³⁵S] Caᵥβ3, [³⁵S] Caᵥβ3-I-II_{2.1} and [³⁵S] Caᵥβ3-I-II_{2.1}ΔAID) are incubated in the presence or in the absence of the GST-AID_{1.2} fusion protein or of the GST protein, and optionally in the presence of increasing amounts of Gβγ (10 to 900 nM, Calbiochem).

The incubation product is separated by polyacrylamide gel electrophoresis (SDS-PAGE) and the gel is autoradiographed.

2) Results

The results illustrated in FIGS. 2 and 3 are as follows:

FIG. 2a shows that the β₃ subunit (1 to 3 pM) binds with the AID_{1.2} domain of the GST-AID_{1.2} fusion protein (1 μM),

FIG. 2b shows that the fusion of the β₃ subunit with the I-II loop of the α2.1 subunit (Caᵥβ₃-I-II2.1 chimera) prevents its binding with the AID_{1.2} domain of the GST-AID_{1.2} fusion protein,

FIG. 2c shows that the deletion of the 18 amino acids of the AID_{2.1} domain (Caᵥβ₃-I-II_{2.1}ΔAID chimera) restores the binding of the β₃ subunit with the AID_{1.2} domain of the GST-AID_{1.2} fusion protein,

FIG. 3 shows that the addition of Gβγ complex displaces the intramolecular interaction between the Caᵥβ subunit and the I-II loop of the α_{2.1} subunit of the Caᵥβ₃-I-II_{2.1} chimera, thus allowing the β₃ subunit to bind with the AID_{1.2} domain of the GST-AID_{1.2} fusion protein; the IC₅₀ concentration of Gpy capable of displacing 50% of the binding between the Caᵥβ subunit and AID_{2.1} domain, after incubation for 30 min at 30° C., is 160 nM; this value is 2 to 3 times higher than those relating to the affinity of Gy for the I-II2.1 loop, previously reported (De Waard et al., Nature, 1997, 385, 446-450).

### EXAMPLE 3

### Demonstration Ex Vivo of the Displacement of the Caᵥα_{2.1}-Caᵥβ Interaction by the G-Protein Bβγ Complex

1) Materials and Methods

a) Cy3-Labeling of the Purified His-Caᵥβ₃ Recombinant Protein

The purified His-Caᵥβ₃ recombinant protein (Geib et al., Biochem J., 2002, 364, 285-292; Fathallah et al., Eur. J. Neurosci., 2002, 16, 219-228) is coupled to monoreactive Cy3 maleimide according to the supplier's instructions (Amersham Pharmacia Biotech).

b) Injection of Xenopus Oocytes and Electrophysiological Recordings

The preparation, the injection of the xenopus oocytes and the electrophysiological recordings are carried out as described in Geib et al., mentioned above. The effects of the Gβγ complexes on the current-voltage relationship and the inactivation of the equilibrium state are analyzed 30 minutes after injection.

c) Fluorescence Transfer (FRET) Measurement

The oocytes are analyzed by confocal microscopy (Leica TCS-SP2 microscope, in the “XYξ” mode), 4 to 7 days after injection.

The fluorescence emission is recorded using an argon laser with an excitation at 488 nm and a dichroic mirror (488/543/633). The fluorescence is measured through 14 filters (10 nm thick) so as to reconstruct the emission spectrum. For each measurement, two different regions are analyzed in order to ensure the reproducibility of the measurement. The FRET levels are estimated through the ratio (585/525) of the fluorescence at 585 nm (Cy3 acceptor emission peak) to the fluorescence at 525 nm (GFP donor emission peak).

2) Results

The Caᵥβ₃ subunit coupled to Cy3 (FIG. 4a) is as active as the Caᵥβ₃ subunit on the regulation of Caᵥα_{2.1} channels expressed in xenopus oocytes (FIG. 4b).

The injection of the Cy3-Caᵥβ₃ or CFP-Caᵥα_{2.1} protein or else of the cDNA encoding said protein, alone or in combination, results in the emission of a high fluorescence signal at the plasma membrane (FIG. 5a).

Analysis of the fluorescence emission between 500 and 640 nm, after excitation at 488 nm (FIG. 5b), shows that GFP-Caᵥα_{2.1} produces a high signal with a maximum at 525 nm, whereas Cy3-Caᵥβ3 alone is relatively nonexcited and produces a weak signal with a maximum at 585 nm. When the two proteins are in the oocytes, the signal emitted at 525 nm decreases drastically, whereas that at 585 nm increases significantly. These changes are readily quantifiable by determining the ratio of the fluorescence signals at 585 nm and 525 nm (R_{f}=0.34±0.03 for GFP-Caᵥα_{2.1} (n=3), R_{f}=1.9±0.10 for Cy3-Caᵥβ₃ (n=3) and R_{f}=3.9±0.22 for GFP-Caᵥα_{2.1}+Cy3-Caᵥβ₃ (n=7)) . Such large changes resulting from a considerable fluorescence transfer demonstrate the proximity of the GFP-Cavα_{2.1} and Cy3-Cavβ3 fluorochromes.

The injection of Gβγ into the ooctyes containing both GFP-Cavα_{2.1} and Cy3-Cavβ₃ induces a rapid disappearance of the fluorescence transfer (FIG. 6). By comparison, the injection of Gβγ has no effect in the cells containing only GFP-Cavα_{2.1} or Cy3-Cavβ₃.

The final ratio of the fluorescence signals (0.82±0.06, n=7) is of the order of that observed in the oocytes containing only GFP-Cavα_{2.1} or Cy3-Cavβ₃, indicating that the dissociation of the Cy3-Cavβ₃ channel is considerable (FIG. 7). By comparison, the injection of heat-inactivated Gβγ has no effect (R_{f}=3.74±0.4, n=3).

These results demonstrate that Gβγ is as capable of displacing, ex vivo, the Cavβ₃ subunit from its site for binding to the Cavα2.1 channel.

### EXAMPLE 4

### Construction of a Biosensor for Measuring the Activity of G Proteins by the Fret Technique

A chimeric protein containing a fluorescence donor fluorophore (EGFP) at its NH₂ end and a fluorescence acceptor fluorophore (CFP) at its COOH end is constructed from the vector pEYFPmemb (Clontech). This vector has the advantage of having:

a GAP-43 sequence that makes it possible to anchor the chimera to the plasma membrane via its NH₂ end. The anchoring to the membrane has the advantage, firstly, of keeping the protein at the membrane and, secondly, of increasing the probability of encounter between the chimeric protein and its Gβγ ligand, which is itself anchored to the membrane via binding of the palmitoylation type, and

an EYFP sequence downstream of GAP-43.

The construction is carried out in two steps:

1st cloning step:
The DNA fragment encoding the GK-like domain of the β subunit (Hanlon et al., FEBS, 1999, 445, 366-370) fused to the I-II loop of the α₁ subunit is amplified by PCR from the plasmid pcDNA3-Caᵥβ3-I-II_{2.1} (Example 1) and is then cloned 3′ of the EYFP gene.

More precisely, the PCR amplification is carried out using the following sense and antisense primers:

BsiW I Pvu I

(SEQ ID NO: 6)

5′-AGCCGTACGCGATCGCATCTCTAGCCAAGCAGAAGCAAA-3′
Hpa I Spe I

(SEQ ID NO: 7)

5′-CCCGTTAACCCCACTAGTCTGAGTTTTGACCATGCGACGGAT-3′

The PCR product obtained is cloned between the BsiW I and Hpa I sites of the plasmid pEYFPmemb, so as to give the plasmid pEYFmemChimBeta3I-II.

2nd cloning step:
The cDNA encoding ECFP is amplified by PCR and then cloned into the above plasmid, 3′ of the β3-I-II insert.

More precisely, the ECFP is amplified by PCR from the vector PECFP (Clontech), using the following sense and antisense primers:

Spe I

(SEQ ID NO: 8)

5′-GGGACTAGTATGGTGAGCAAGGGCGAGGAGCTG-3′
Hpa I

(SEQ ID NO: 9)

5′-CCCGTTAACTGCCGAGAGTGATCCCGGCGGCGGT-3′

The PCR product obtained is cloned between the Spe I and Hpa I sites of the plasmid pEYFmemChimBeta3I-II, to give the plasmid pCHIC corresponding to the sequence SEQ ID NO: 10.

1. A chimeric protein derived from a high-threshold calcium channel, characterized in that it comprises at least one β subunit or a fragment thereof including at least the BID domain, fused, at its NH₂ or COON end, with the I-II loop of an α₁ subunit or fragment thereof including at least the AID domain.

2. The chimeric protein as claimed in claim 1, characterized in that it consists of a β subunit fused, at its NH₂ or at its COOH end, with the I-II loop of an α₁ subunit.

3. The chimeric protein as claimed in claim 1, characterized in that it consists of the GK-like domain of a β subunit fused, at its NH₂ or COOH end, with the I-II loop of an α₁ subunit.

4. The protein of claim 1, characterized in that the β subunit, or a fragment thereof, and the I-II loop, or a fragment thereof, are separated by a spacer peptide.

5. The chimeric protein of claim 1, characterized in that it is derived from a G-protein-sensitive high-threshold calcium channel.

6. The chimeric protein as claimed in claim 5, characterized in that it comprises the I-II loop of an α₁ subunit selected from α_{1A}, α_{1B}, and α_{1E}, or a fragment thereof.

7. The chimeric protein as claimed of claim 1, characterized in that it comprises a β subunit selected from the group consisting of β₁, β₂, β₃ and β₄, or a fragment thereof.

8. A variant chimeric protein derived from a chimeric protein as claimed in claim 1, characterized in that it has a mutation of at least one amino acid in the sequences of said β subunit and/or of the I-II loop of an α₁ subunit.

9. The variant chimeric protein as claimed in claim 8, characterized in that said mutation modifies the affinity of the β subunit for the fragment of the I-II loop of the α subunit and/or vice versa.

10. The variant chimeric protein as claimed in claim 8, characterized in that said mutations are selected from the following mutations of the AID domain of the I-II loop of the α₁ subunit: Q383A, Q384A, E386D, E386S, L389H, G391R, Y392S, Y392F, W395A, 1396A and E400A.

11. The chimeric protein as claimed in claim 1, characterized in that it is coupled, to at least one suitable label allowing the detection and/or the purification and/or the immobilization of said protein.

12. The chimeric protein as claimed in claim 11, characterized in that it comprises an acceptor or donor fluorophore respectively at its NH₂ and/or COOH end.

13. The chimeric protein as claimed in claim 12, characterized in that the acceptor fluorophore is the fluorescent protein CFP or BFP and the donor fluorophore is the fluorescent protein GFP or YFP.

14. A peptide, characterized in that it comprises a fragment of at least 7 amino acids of the sequence of the chimeric protein as claimed in claim 1, which fragment includes at least the 7 amino acids located at the junction of the β subunit and of the I-II loop of the α₁ subunit of a calcium channel.

15. An antibody, characterized in that it is directed against a peptide as claimed in claim 14.

16. A nucleic acid molecule, characterized in that it is selected from the sequences encoding a chimeric protein as claimed in claim 1, and the sequences complementary to the above sequences, that may be sense or antisense.

17. Probes and primers, characterized in that they comprise a sequence of approximately 10 to 30 nucleotides corresponding to that located at the junction of the β subunit and of the I-II loop of the a₁ subunit of a calcium channel or of their fragments as defined in claim 1.

18. Primers capable of amplifying the β subunit and/or the I-II loop of the α₁ subunit of a calcium channel or their fragments as defined in claim 1, characterized in that they are selected from the group consisting of the sequences SEQ ID NO: 1, 2, 4, 6, 7, 8 and 9.

19. A recombinant vector, characterized in that it comprises an insert selected from the group consisting of the nucleic acid molecules as claimed in claim 16.

20. The recombinant vector as claimed in claim 19, characterized in that it is a eukaryotic expression vector having a sequence selected from the group consisting of the sequences SEQ ID NO: 5 and SEQ ID NO: 10.

21. A cell modified with a recombinant vector as claimed in claim 19.

22. The modified cell as claimed in claim 21, characterized in that it is a eukaryotic cell.

23. The modified cell as claimed in claim 21, characterized in that it expresses at least one receptor capable of coupling to G proteins.

24. A nonhuman transgenic mammal, characterized in that all or some of its cells are transformed with a nucleic acid molecule as claimed in claim 16.

25. The use of the product of claim 1 for studying the G-protein-coupled receptor-dependent cell signaling and regulatory pathways.

26. The use of the product selected from claim 1 for screening agonists and/or antagonists of G-protein-coupled receptor-dependent cell signaling and regulatory pathways.

27. The use of the product of claim 1, for screening antagonists of the interaction between the α₁ and β subunits of high-threshold calcium channels.

28. A method for studying the G-protein-coupled receptor-dependent cell signaling and regulatory pathways, characterized in that it comprises at least the following steps:
a₁) culturing of modified cells expressing a chimeric protein derived from a G-protein-sensitive calcium channel and a G-protein-coupled receptor, as claimed in claim 23,
b₁) transduction of a signal via said G-protein-coupled receptor, by any appropriate means, and
c₁) determination, by any appropriate means, of the proportion of said chimeric protein expressed in said cells that is bound to a Gβγ subunit.

29. A method for screening agonists/antagonists of the G-protein-coupled receptor-dependent cell signaling and regulatory pathways, characterized in that it comprises at least the following steps:
a₂) culturing of modified cells expressing a chimeric protein derived from a G-protein-sensitive calcium channel and a G-protein-coupled receptor, as claimed in claim 23,
b₂) transduction of a signal via said G-protein-coupled receptor, by any appropriate means,
c₂) comparative determination, by any appropriate means, of the proportion of said chimeric protein expressed in the cells that is bound to a Gβγ subunit, before and after the bringing into contact of said cells in b₂) with a molecule to be tested, and
d2) identification of the molecules that are agonists/antagonists of the G-protein-coupled receptor-dependent cell signaling and regulatory pathways, corresponding to those capable respectively of increasing and of decreasing the cellular concentration of free Gβγ subunits.

30. The method as claimed in claim 28, characterized in that said modified cells in a₁) or in a₂) express a chimeric protein coupled, at its NH₂ and COOH ends, respectively to a fluorescence donor fluorophore and a fluorescence acceptor fluorophore, and said determination in c₁) or in c₂) is carried out by means of the fluorescence transfer (FRET) technique.

31. A method for screening antagonists of the interaction between the α₁ and β subunits of high-threshold calcium channels, characterized in that it comprises at least the following steps:
a₃) bringing a molecule to be tested into contact with a chimeric protein derived from a G-protein-sensitive or -insensitive calcium channel as claimed claim 1 and with a peptide comprising the AID domain of a G-protein-insensitive α₁ subunit,
b₃) measuring, by any appropriate means, the binding of said chimeric protein to said peptide, and
c₃) identifying the antagonists of the interaction between the α₁ and β subunits corresponding to those with which binding of said chimeric protein to said peptide is observed.

32. The screening method as claimed in claim 31, characterized in that said peptide comprising the AID domain is immobilized on a solid support and said chimeric protein is a chimeric protein.

33. A kit for implementing a method as claimed in any claim 28, characterized in that it comprises at least one product selected from the group consisting of chimeric proteins, nucleic acid molecules, recombinant vectors, modified cells and the nonhuman transgenic mammals.
